(19) United States
(12) Patent Application Publication
(10) Pub. No.: US 2013/0224837 A1
(43) Pub. Date: Aug. 29, 2013

(54) INOCULATION TOOL AND METHOD OF USE THEREOF

(71) Applicants: Margret Elaine Leventhal, Berkeley, CA (US); Kent Frank McCue, El Cerrito, CA (US)

(72) Inventors: Margret Elaine Leventhal, Berkeley, CA (US); Kent Frank McCue, El Cerrito, CA (US)

(21) Appl. No.: 13/405,045

(22) Filed: Feb. 24, 2012

(51) Int. Cl. C12M 1/26; C12N 1/00

(52) U.S. Cl. 435/243; 435/309.1

(57) ABSTRACT

The present invention describes disposable inoculation tools designed to fit into an appropriately sized test tube with the lid closed. A method that reduces the overall number of steps required to complete inoculation and can reduce repetitive motion injuries that might result from repetitive inoculation is also disclosed.

### BACKGROUND OF THE INVENTION

1. Field of the Invention

This invention relates to a tool and method, in particular an inoculation tool and its method of use.

2. Description of Related Art

Inoculation is the placement of a material into surroundings that are suitable for a particular purpose. That purpose may include, but is not limited to, growing, reproducing, storing, transporting or testing that material. For example, the material can be introduced into surroundings suited to its growth, such as a culture medium. The material can also be implanted in a person, animal, plant, food, or beverage to produce a disease for study or to stimulate disease resistance.

A variety of tools are used to manipulate a portion of microbial colonies, culture medium, biological and non-biological samples, plaque(s) and other material samples, which can take any form, for example, a liquid or solid. The purpose of the manipulation may include, but is not limited to, inoculation streaking or archiving. A loop, needle or pick is used to come in contact with the material, after which the tool is considered to be “inoculated”. The inoculated tip is either swiped across a plate, swirled into liquid media, stabbed into some solid media, or used to infect a plant, animal or person, or mixed with another microorganism, food or beverage, or otherwise manipulated.

The existing tools can be, for example, plastic inoculation loops, needles, picks or wooden toothpicks. These tools can be range in length from 3 inches to 11 inches. Typically, but not always, inoculation loops, needles and picks are made of plastic and are pre-sterilized and packaged either individually, in small packs of multiple units or in bulk. Toothpicks are made of wood, bamboo or plastic and may or may not be sterile. Toothpicks are often autoclaved for sterilization. The sterilized toothpicks are transported for use at another location, often but not always a biological hood, where they are usually used once and disposed of Similarly, pre-sterilized tools are opened in the biological hood or other location and are typically used once and disposed of Occasionally, used toothpicks or other tools are collected and autoclaved for reuse.

The existing tools and toothpicks require multiple steps when used. The tool is selected, it comes in contact with the sample material, and the tip is either swiped across a plate, swirled into liquid media, stabbed into some solid media, or used to infect a plant, animal or person, or mixed with another microorganism, food, or beverage or otherwise manipulated. One problem with the inoculation tools of the prior art is that they do not fit within the smaller sized test tubes that are often used in a laboratory. The 0.6 milliliter (ml), 1.5 ml or 2.0 ml test tubes are often utilized because the smaller test tubes can be transferred to a micro centrifuge to separate the solids that sink to the bottoms of the tubes from the liquids that rise to the top of the tubes. The solids and/or liquids can then be used in down stream testing. Thus, one cannot close the lids of test tubes over the existing inoculation tools in order to protect the culture from outside contaminants, evaporation during growth, sealing for long-term storage, or accidental loss of culture during centrifuging and prior to harvesting.

For inoculation tools used in the art, the pick must be broken in half prior to placing the half in the 0.6, 1.5 or 2.0 ml test tube. This can be a clumsy procedure because it is normally done under a biological hood, and can be especially difficult to manipulate once one end of the inoculation tool has already been inoculated. The inoculated half is then placed in a 0.6, 1.5 or 2.0 ml test tube and the lid of the test tube is closed. The final step is the disposal of the tool. These steps can be performed hundreds of times a day and lead to repetitive motion injuries. What is needed is an inoculation tool and method that eliminates the step of the inconvenient breaking of the inoculation tool.

To attempt to solve this problem, scientists will place an inoculated pick in its entirety in a larger 15 ml screw cap tube containing the growth media and screw the cap closed. When the organism and/or spent medium is ready for harvesting, the culture must be transferred from the 15 ml tube to a 0.6, 1.5 or 2.0 ml test tube for bench top centrifugation. This approach eliminates the clumsy toothpick breaking process but adds a time-consuming step of transferring the culture from the 15 ml to the 0.6, 1.5 or 2.0 ml test tube. This method also increases the overall cost of consumables by adding the 15 ml tube to the process. Considering that the above steps can be repeated hundreds of times each day, the amount of time and money spent during the process can be greatly reduced using an improved inoculation tool to solve these problems in the art.

What is needed is an inoculation tool and method that is more user- friendly, less time consuming, and reduces the number of consumables required to complete the process. With these goals in mind, the inventor created an improved inoculation tool and method that reduce the number of steps required to complete the inoculation, reduce repetitive motion injuries that might result from such repetitive activities, and reduce the time it takes to complete the required tasks.

### BRIEF SUMMARY OF THE INVENTION

The present invention comprises an inoculation tool having a terminating end, a stalk region, and a tip. The tool can have a length ranging from 0.59 inches to 1.6 inches and a width ranging from 0.010 to 0.315 inches.

In a preferred embodiment, the length ranges from 1.260 inches to 1.300 inches from the terminating end to the tip, and a distance that is at least 0.250 inches from the tip and at most 0.375 inches from the tip has a first width that ranges from 0.093 inches to 0.107 inches. The first width narrows toward the tip to a second width that ranges from 0.011 to 0.068 inches. The tip can comprise a flat end or a pointed end. In another preferred embodiment, the tool has a length of 1.280 inches from the terminating end to the tip, and at a distance that is 0.312 inches from the tip, the width is 0.10 inches. The width narrows toward the tip to a second width of 0.063 inches. The tip can be formed to have a flat end.

In another preferred embodiment, the tool has a length of 1.280 inches from the terminating end to the tip, and at a distance that is 0.312 inches from the tip, the width is 0.10 inches. The width narrows toward the tip to a second width of 0.016 inches. The tip can be formed to have a pointed end.

The inoculation tool can be made of a biodegradable material. The tool can have one or more ridges to improve handling of the tool. Also to improve handling, besides having a round and/or conical shape, the tool can have a star-shaped cross-section and a flat-sided geometric cross-section. Both ends of the tool can have a flat, round, narrowed, or angled shape. The tool can be pre-sterilized.

In addition, an improved inoculation method using the inoculation tool is disclosed.

### BRIEF DESCRIPTION OF THE SEVERAL VIEWS OF THE DRAWINGS

FIGS. 1A-1J illustrates different embodiments of an inoculation tool in accordance with the present invention;

FIG. 2A illustrates a preferred embodiment of an inoculation tool in accordance with the present invention;

FIG. 2B illustrates a preferred embodiment of an inoculation tool in accordance with the present invention; and

FIG. 3 illustrates a system comprising an inoculation tool in accordance with the present invention.

### DETAILED DESCRIPTION OF THE INVENTION

The detailed description set forth below in connection with the appended drawings is intended to provide example embodiments of the present invention and is not intended to represent the only forms in which the invention may be constructed or utilized. The description sets forth the functions and the sequences of steps for constructing and operating the invention. However, it is to be understood that the same or equivalent functions and sequences may be accomplished by different embodiments that are also intended to be encompassed within the spirit and scope of the invention.

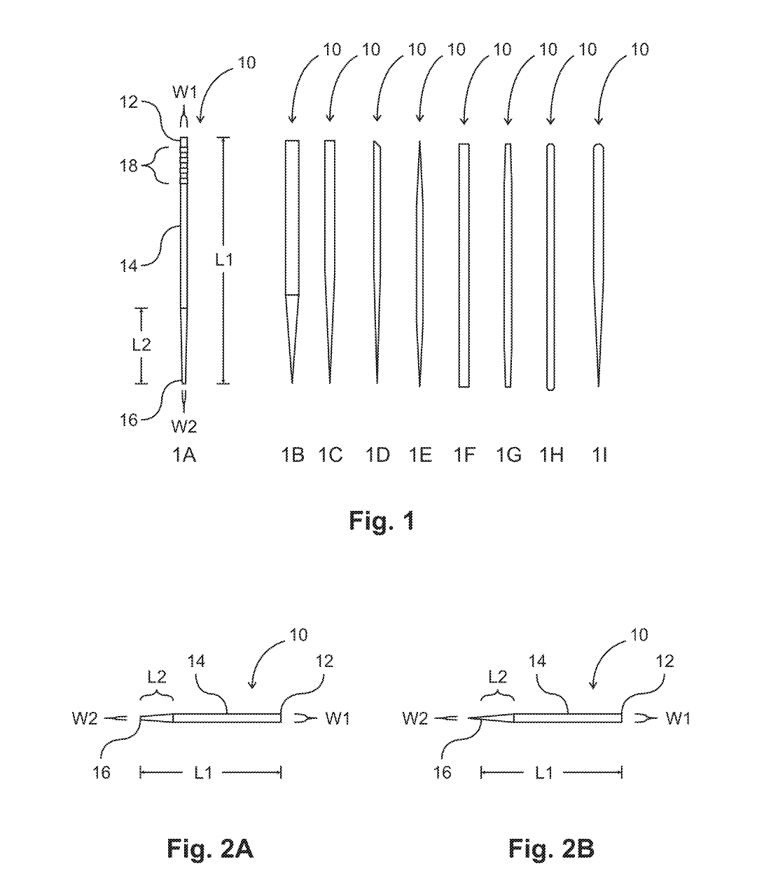

Some embodiments of the invention will be described in detail with reference to FIGS. 1-3. Additional embodiments, features, and/or advantages of the invention will become apparent from the description or may be learned by practicing the invention. The drawings in the figures are not necessarily drawn to scale and have like numerals referring to like features through both the drawings and the description.

Described herein are disposable inoculation tools 10 that have been custom designed to fit into 0.6, 1.5, and/or 2.0 ml micro tubes with the lid closed. The inoculation tool may be comprised of any appropriate material including wood, bamboo, or any type of plastic including but not limited to: PET, PP, PC, PMMA, PETG, PVC, PE, PS, PA, ABS, EVA, EPS, HIPS, SAN, RPET, CPET, APET, TPU, TPR, Synthetic Butyl Rubber, Polyesters, homopolymers and Copolymers. The plastic used may also contain an additive to give the tool a desired quality. Examples of such additives are, but are not limited to: additives known in the art that give a hydrophobic quality to the tool and additives known in the art that alter the chemical bonds in the plastic to accelerate decomposition of the plastic in the waste stream, giving the inoculation tool a more environmentally ecological quality. The inoculation tool 10 can be sold in sterile packs or non-sterile packs. The inoculation tool 10 can be pointed, cut at a 90° angle, or at any other angle, at one end or at both ends 12, 16. The inoculation tools 10 shown in FIG. 1 are round and/or conical, or a combination thereof, in shape; however, they can be round, flat, triangular, star-shaped, or have any other cross sectional form. The tool 10 can have ridges 18 that facilitate gripping, or it can be smooth. The tool 10 can be colored or it can be the natural color of the material the tool 10 is made from.

FIGS. 1A-1J illustrate ten embodiments of the inoculation tool 10. The inoculation tool 10 can be comprised of a terminating end 12, a stalk region 14, and a tip 16. The length L1 of the entire inoculation tool 10, from the terminating end 12 to the tip 16 can range in length from approximately 0.59 to 1.6 inches, and is preferably 1.26 to 1.30 inches. A first width of the stalk region 14 and/or terminating end 12 is labeled W1 in the figures. A second width of the tip 16 is labeled W2 in the figures. The widths W1 and W2 can range from 0.010 to 0.315 inches thick. The first width W1 of the stalk region 14 and/or terminating end 12 can be the same as or different from the second width W2 of the tip 16. At a distance L2 from the tip 16 of the tool 10, the first width W1 of the tool 10 can gradually narrow to a second width W2 at the tip 16 of the tool 10. Alternatively, the tool can have a first width W1 at the terminating end 12 and gradually narrow to a second width W2 at the tip 16 of the tool 10.

FIG. 1A shows an inoculation tool 10 that is approximately 1.280 inches long and 0.10 inches wide at its widest end, the terminating end 12. The tool 10 in FIG. 1A is cross-sectionally round from the terminating end 12 along the stalk region 14 to a position at approximately 0.968 inches from the terminating end 12, which is approximately 0.312 inches from the tip 16. At approximately 0.312 inches from the tip 16 and approximately 0.968 inches from the terminating end 12, the stalk region 14 begins to gradually narrow towards the end of the tip 16. FIG. 1A shows the tip 16 narrowing to 0.063 inches and cut perpendicular to the longitudinal axis of the tool 10 and ending in a flat round surface having a diameter of 0.063 inches. FIG. 1A shows optional ridges 18 that are formed in the stalk region 14 of the inoculation tool 10 to aid in grasping. In the non-limiting embodiment shown there are four ridges 18. The ridges 18 can be the same size or different sizes and can be of the same depth or different depths. There can be no ridges 18 or up to and including as many ridges 18 as needed along the entire length of the stalk region 14.

FIG. 1B shows a wider version of the tool 10. FIGS. 1B, 1C, 1F and 1G show the inoculation tool without the ridges 18 having a cut at the terminating end 12 of the stalk region 14 that is perpendicular to the longitudinal axis of the tool 10 such that the terminating end 12 comprises a flat end with a cross-sectional round surface. FIG. 1D shows the terminating end 12 of the stalk region 14 cut at an angle. Angular cuts can be between 1° and 90°. FIG. 1E shows the inoculation tool 10 with both ends 12, 16 terminating to a pointed tip. FIG. 1F shows the inoculation tool with both ends 12, 16 terminating in a cut perpendicular to the longitudinal axis of the tool 10. FIG. 1G shows the inoculation tool with both ends 12, 16 terminating in a perpendicular cut but the stalk region 14 tapering towards these ends. FIG. 1H shows the inoculation tool with both ends 12, 16 terminating in a round-shaped form. FIG. 1I shows the terminating end 12 of the stalk region 14 to be rounded while the tip 16 is pointed. FIG. 1J shows the tool 10 can have a conical shape from a first width W1 at the terminating end 12 and gradually narrowing to a second width W2 at the tip 16 of the tool 10. Any and all combinations of the above terminating end 12, stalk region 14, and tip 16 can be formed.

A preferred embodiment of the inoculation tool is shown in FIG. 2. In preferred embodiments, the length L1 of the inoculation tool 10 can range from 1.26 inches to 1.30 inches. As shown in FIG. 2A, the length L1 is 1.280 inches. The preferred length L2 can range from 0.250 to 0.375 inches. The length L2 in FIG. 2A is 0.312 inches. The preferred width W1 of the tool can range from 0.093 to 0.107 inches and the preferred width W2 can range from 0.058 to 0.068 inches. As shown in FIG. 2A, the width W1 of the terminating end 12 of the tool 10 is 0.10 inches and the width W2 of the tip 16 of the tool 10 is 0.063 inches and terminates in a flat round end. A second preferred embodiment of the inoculation tool is shown in

FIG. 2B. In preferred embodiments, the length L1 of the inoculation tool 10 can range from 1.26 inches to 1.30 inches. As shown in FIG. 2B, the length L1 is 1.280 inches. The preferred length L2 can range from 0.250 to 0.375 inches. The length L2 in FIG. 2B is 0.312 inches. The preferred width W1 of the tool can range from 0.093 to 0.107 inches and the preferred width W2 can range from 0.011 to 0.021 inches. As shown in FIG. 2B, the width W1 of the terminating end 12 of the tool 10 is 0.10 inches and the width W2 of the tip 16 of the tool 10 is 0.016 inches and terminates in a pointed tip.

Embodiments having a tip 16 with a pointed end can allow for a very detailed extraction of material. Embodiments having a tip 16 with a flat-surfaced end, and thus a relatively larger second width W2 at the tip 16 than a pointed end embodiment, can extract a larger amount of material. The terminating end 12 having a first width W1 can be used to extract even more material.

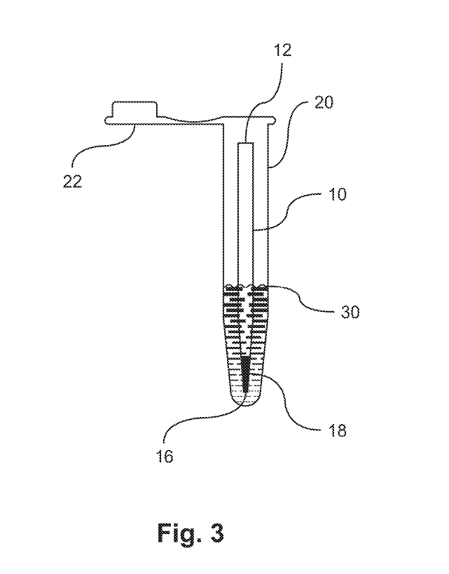

A system comprising the inoculation tool 10 and a test tube 20 is shown in FIG. 3. A method of using the inoculation tool 10 comprises one or more of the steps of selecting the inoculation tool 10, contacting a sample material 18 with the inoculation tool 10, and dropping the tool 10 into appropriately sized test tube 20, wherein the test tube 20 has a lid 22 and the inoculation tool 10 is enclosed in its entirety when the test tube 20 is subsequently closed. The test tube 20 can be any size, such as a micro tube, and further comprise an appropriate media 30 for the chosen purpose, in which the inoculated end 16 of the inoculation tool 10 having the sample material 18 is placed. The test tube 20 can be closed with a lid 22 that is attached or not attached to the test tube. The lid 22 can be attached to the test tube 20 in any manner known in the art.

The present invention eliminates the inconvenient step of breaking the inoculation tool because the tool fits in its entirety into a 0.6, 1.5 or 2.0 ml test tube. The invention also does not require that the inoculation procedure to be performed in a larger test tube that is later transferred to a micro tube, which reduces the number of consumables required to complete the process and reduces risks of contamination. When using the larger test tube method the user risks an incomplete transfer of the media and the inoculation may not take place. This method using the improved inoculation tool 10 is an improvement for the user as it reduces the overall number of steps required to complete the tasks, takes less time, is more likely to achieve a successful inoculation, and can reduce repetitive motion injuries that might result from such repetitive activities.

The described inoculation tool 10 is applicable in any situation where a user wishes to manipulate a portion of microbial colonies, biological and non-biological samples, plaque(s), liquids/solids, and other material samples. It is particularly useful to facilitate the movement of a small sample or inoculate from the sample to a micro tube or other receiving vessel.

While the present invention has been described with regards to particular embodiments, it is recognized that additional variations of the present invention may be devised without departing from the inventive concept. A person skilled in the art would appreciate that exemplary embodiments described hereinabove are merely illustrative of the general principles of the present invention. Other components, configurations, modifications or variations may be employed that are within the scope of the invention. Accordingly, the drawings and description are illustrative and not meant to be a limitation thereof.

What is claimed is:

1. An inoculation tool comprising:
a stalk region having a terminating end and a tip;
said tool has a length that ranges from 0.59 inches to 1.6 inches from said terminating end to said tip and a width that ranges from 0.010 to 0.315 inches.

2. The inoculation tool of claim 1, said length ranges from 1.26 inches to 1.30 inches from said terminating end to said tip, wherein at a distance on said stalk region that is at least 0.250 inches from said tip and at most 0.375 inches from said tip said stalk region has a first width that ranges from 0.093 inches to 0.107 inches, said first width narrows toward said tip to a second width that ranges from 0.011 to 0.068 inches.

3. The inoculation tool of claim 2, wherein said tool has a length of 1.280 inches from said terminating end to said tip, wherein said stalk region has a first width of 0.10 inches at a distance that is 0.312 inches from said tip, said first width narrows toward said tip to a second width of 0.063 inches.

4. The inoculation tool of claim 3, wherein said tip has a flat end.

5. The inoculation tool of claim 2, wherein said tool has a length of 1.280 inches from said terminating end to said tip, wherein said stalk region has a first width of 0.10 inches at a distance that is 0.312 inches from said tip, said first width narrows toward said tip to a second width of 0.016 inches.

6. The inoculation tool of claim 5, wherein said tip has a pointed end.

7. The inoculation tool of claim 1, said length ranges from 1.26 inches to 1.30 inches from said terminating end to said tip, wherein said stalk region has a first width that ranges from 0.093 inches to 0.107 inches and a second width that ranges from 0.011 to 0.068 inches.

8. The inoculation tool of claim 1, wherein said inoculation tool is comprised of a biodegradable material.

9. The inoculation tool of claim 1, said stalk region further comprises one or more ridges.

10. The inoculation tool of claim 1, said tool has a cross-section comprising a shape selected from the group of a circular cross-section such that the tool has a conical shape, a star-shaped cross-section, and a flat-sided geometric cross-section.

11. The inoculation tool of claim 1, said terminating end has a shape selected from the group consisting of a flat end, a round end, a narrowed end, a pointed end, and an angled end.

12. The inoculation tool of claim 1, said tip has a shape selected from the group consisting of a flat end, a round end, a narrowed end, a pointed end, and an angled end.

13. The inoculation tool of claim 1, wherein said tool is sterilized.

14. A method of using the inoculation tool of claim 1, comprising the steps of:
contacting a sample material with said inoculation tool;
placing said inoculation tool into a test tube having a culture media inside said test tube and a lid; and
closing said test tube with said lid, whereby said inoculation tool is enclosed in its entirety in said test tube.

15. The method of claim 14, wherein said test tube is selected from the group consisting of a 0.6 ml tube, 1.5 ml tube, and a 2.0 ml tube.

16. An inoculation system comprising:
said inoculation tool of claim 2; and
a test tube having a lid, wherein said inoculation tool can be enclosed in its entirety in said test tube when said lid covers said test tube.

17. The system of claim 16, wherein said test tube is selected from the group consisting of a 0.6 ml tube, 1.5 ml tube, and a 2.0 ml tube.

18. An inoculation system comprising:
said inoculation tool of claim 7; and
a test tube having a lid, wherein said inoculation tool can be enclosed in its entirety in said test tube when said lid covers said test tube.

19. The system of claim 18, wherein said test tube is selected from the group consisting of a 0.6 ml tube, 1.5 ml tube, and a 2.0 ml tube.
